(19) United States
(12) Patent Application Publication
(10) Pub. No.: US 2007/0218087 A1
(43) Pub. Date: Sep. 20, 2007

(54) Method of controlled release of vanillin for nasal and/or pulmonary uptake

(76) Inventor: Norbert Hoenzelaer, Kleve-Rindern (DE)

Correspondence Address: LERNER GREENBERG STEMER LLP, P O BOX 2480, HOLLYWOOD, FL, 33022-2480, US

(21) Appl. No.: 11/804,842

(22) Filed: May 21, 2007

Related U.S. Application Data
(63) Continuation-in-part of application No. 11/084,349, filed on Mar. 18, 2005.
Application No. 11/084,349 is a continuation of application No. PCT/EP02/13820, filed on Dec. 6, 2002.

(30) Foreign Application Priority Data
Dec. 7, 2001 (DE) 101 60 110.8

(51) Int. Cl. A61K 9/00

(52) U.S. Cl. 424/400

(57) ABSTRACT

A gaseous vanillin compound is continuously released for nasal and/or pulmonary uptake. The gaseous vanillin compound is provided in a patch having a layer that is impermeable to the gaseous vanillin compound, a layer permeable to the gaseous vanillin compound, and the vanillin localized between the two layers. The patch is placed on the person so as to stimulate the release of serotonin in the central nervous system and for utilizing the patch to reduce food cravings of the person in need thereof.

### CROSS-REFERENCE TO RELATED APPLICATION

This is a continuation-in-part of copending application Ser. No. 11/084,349, filed Mar. 18, 2005, which was a continuing application, under 35 U.S.C. § 120, of co-pending international application No. PCT/EP02/13820, filed Dec. 6, 2002, which designated the United States; this application also claims the priority, under 35 U.S.C. § 119, of German patent application No. 10160110.7, filed Dec. 7, 2001; the prior applications are herewith incorporated by reference in their entirety.

### BACKGROUND OF THE INVENTION

### Field of the Invention

The present invention relates to a device for the controlled release of vanillin, ethylvanillin derivatives or mixtures thereof, to a process for producing said device, to a device obtainable by said process, to the use of a composition comprising vanillin, ethylvanillin or mixtures thereof, and to the use of the device.

Numerous aromas develop pronounced effects on various receptors of neurotransmitters (GABA, NMDA, KA, nACh). Fragrances such as geraniol (“rose”) and butyl acetate (“fruit”) stimulate the GABA receptor. In high doses, receptor stimulants of this kind even have a hypnotic and anesthetizing effect. Vanillin, by contrast, similarly to caffeine, inhibited said receptor. Since, according to current understanding, the most important physiological effects of caffeine are based on this inhibition, vanillin could have a similar stimulant effect to caffeine. Vanillin also inhibited the nicotinic acetylcholine receptor (nACh), a property which it shares with the snake poison cobratoxin or the arrow poison curare. Vanillin also had an inhibitory effect on the glutamate receptors (NMDA, KA). This effect is comparable in principle with that of phencyclidine, which is also known as a narcotic (“Angel dust”).

As well as this receptor inhibition, in the central nervous system vanillin also influences the craving to consume food. In this context it is supposed that the vanillin is able to increase the concentration of the neurotransmitter serotonin in the brain (see for example Williams, J. Psychopharmacol 1998, 12(2): 115-21). Increased brain serotonin concentration, however, leads demonstratively to a reduced craving to consume food.

Since serotonin cannot be prepared synthetically, and since there is in any case virtually no possibility of uptake of said neurotransmitter into the brain, owing to the compartment systems located there, attempts have now been made to obtain an increase in brain serotonin concentration through the use of the fragrance vanillin.

Uptake of this fragrance via the blood-brain barrier is possible on account of its lipophilic properties. A second possible uptake pathway, besides that of the blood-brain barrier, is the olfactory nerve (bulbus olfactorius), which allows transneural transport into the brain (Lloyd Hastings: “Sensory neurotoxicology: Use of the olfactory system in the assessment of toxicity”, in: Neurotoxicology and Teratology 1990/12/pp. 445-9). Vanillin, accordingly, is able to enter the central nervous system via the nose. In the patent literature, accordingly, devices have been numerously described by means of which this fragrance can be vaporized in the vicinity of the body and so breathed in continuously (uptake of vanillin via the olfactory nerve) or with which vanillin can be supplied continuously to the circulation (uptake via the blood-brain barrier).

European patent application EP A 645 081, for example, describes an insect control device comprising a container which is covered by a film impervious to gaseous essential oils. Following removal of a protective layer, the insecticidal compositions contained in said container emerge from the container continuously through the film. The disadvantage of these containers is that they are not suitable for continuous release of vanillin in the vicinity of the body.

To avoid this disadvantage, international PCT publication WO 98/17262 A1 describes a device in the form of a patch, which is intended for transdermal administration of the active substance and the active substance is applied in a lint-like material. However, transdermal administrations needs official permissions. Furthermore, lint-type materials limit the carrying comfort.

Likewise, international PCT publication WO 00/16752 describes a device for transdermal administration of the active substance, in which the active substance(s) are applied in a “matrix-layer”, which has a thickness of 1 mm to 10 mm, which again limits the carrying comfort.

To avoid this disadvantages, European published patent application EP A 844 872 (corresponding to WO 97/03658 and US 2003/0012811 A1) describes a device for administering vanillin that comprises lint or a similar material impregnated with the vanillin-releasing substance, the lint being contained in a self-adhesive patch for application to the skin, and the exposed surface of the patch having a central hole through which the vanillin is able to emerge from the lint. The disadvantage of this device, however, is that the rate at which the vanillin emerges cannot be adequately regulated.

Last but not least U.S. Pat. No. 3,996,934 discloses nasal strips and dilators adhesively applied to the human nose, which substantially prevents a nasal wall tissue of a nose from drawing in during breathing.

### SUMMARY OF THE INVENTION

The object on which the present invention is based was to overcome the disadvantages arising from the prior art.

The object of the invention, moreover, was to provide a device by means of which it is possible to release vanillin continuously in the vicinity of the body so that it can be taken up via the nose.

The above objects have been achieved by means of a device for continuously releasing at least one vanillin compound such as vanillin itself (3 methoxy-4 hydroxybenzaldehyde) or at least one vanillin derivative, preferably ethylvanillin (3 ethoxy-4 hydroxybenzaldehyde), or mixtures thereof, comprising a layer impermeable to the at least one gaseous vanillin compound, a layer permeable to the at least one gaseous vanillin compound, and a composition comprising the at least one vanillin compound which is located between these two layers. The device is used in the vicinity of the body of a wearer, preferably by adhesion to the skin of the wearer. The wearer is preferably a mammal.

The layer impermeable to the at least one gaseous vanillin compound is preferably a film comprising polyester, polypropylene, polyethylene, ethylene-propylene copolymers, polyamides, such as nylon 6,6, metals, such as aluminum, polyethylene, poly(tetrafluoroethylene), polycarbonate, polyethylene terephthalate, polybutyrate, polyurethane or polyvinyl chloride, particular preference being given to a film comprising polypropylene or polyethylene or to a film comprising an ethylene-propylene copolymer. Among these films, very particular preference is given to polyethylene films which are obtainable under the commercial designation Sclairfilm® from DuPont, Canada. The thickness of the layer impermeable to the at least one gaseous vanillin compound is preferably chosen so that within a time interval of 24 hours not more than 1%, preferably not more than 0.1%, and more preferably not more than 0.01% by weight of the composition can evaporate via this film at room temperature under a pressure of 1 bar. Preferably this layer has a thickness in a range from 0.001 to 1 mm, more preferably in a range from 0.01 to 0.5 mm, and with further preference in a range from 0.1 to 0.25 mm.

The layer permeable to the at least one gaseous vanillin, gaseous vanillin derivative or mixtures thereof is preferably a film that has the property whereby within a time interval of 24 hours at least 10%, preferably at least 25%, and more preferably at least 50% by weight of the vanillin, ethyl vanillin or mixtures thereof that are present in the composition can evaporate via this film at room temperature under a pressure of 1 bar. Preferred films are films comprising polyethylene, polyamide, ethylene-vinyl acetate copolymer or mixtures of these monomers. The permeable layer preferably has a thickness in a range from 0.001 to 1 mm, more preferably in a range from 0.01 to 0.5 mm, and with further preference in a range from 0.1 to 0.25 mm. It is further preferred in this context that via the choice of material of the permeable layer and its thickness the amount of the composition evaporated through this layer in a defined time interval at a defined temperature and under a defined pressure can be regulated. In one particular embodiment of this device the permeable layer has microscopic holes having a diameter in a range between 1 and 100 Å, more preferably in a range between 10 and 50 Å, which is preferably obtainable by bombarding the layer with heavy atoms, such as with barium atoms, for example. The density of holes in the film, indicated in the number of holes per mm², and the diameter of the holes likewise make it possible to regulate the amount of composition evaporated through the permeable layer within a defined time interval at a defined temperature and under a defined pressure.

It is further preferred in this context for both the permeable layer and the impermeable layer to have a thickness of not more than 0.1 mm, more preferably of not more than 0.01 mm, so that the device of the invention is not rigid and is able when placed onto an uneven surface to conform to that surface.

Other features which are considered as characteristic for the invention are set forth in the appended claims.

Although the invention is illustrated and described herein as embodied in a device and patch for controlled release of vanillin, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE DRAWINGS

FIG. 1 shows an embodiment of the device of the invention in the form of a patch as a cross section. With this patch, continuous uptake of the at least one vanillin compound nasally or pulmonarily is possible;

FIG. 2 shows the patch according to FIG. 1 without any further additional packaging; and

FIG. 3 shows the patch according to FIG. 2, applied to the upper chest area of a human wearer.

### DESCRIPTION OF THE PREFERRED EMBODIMENTS

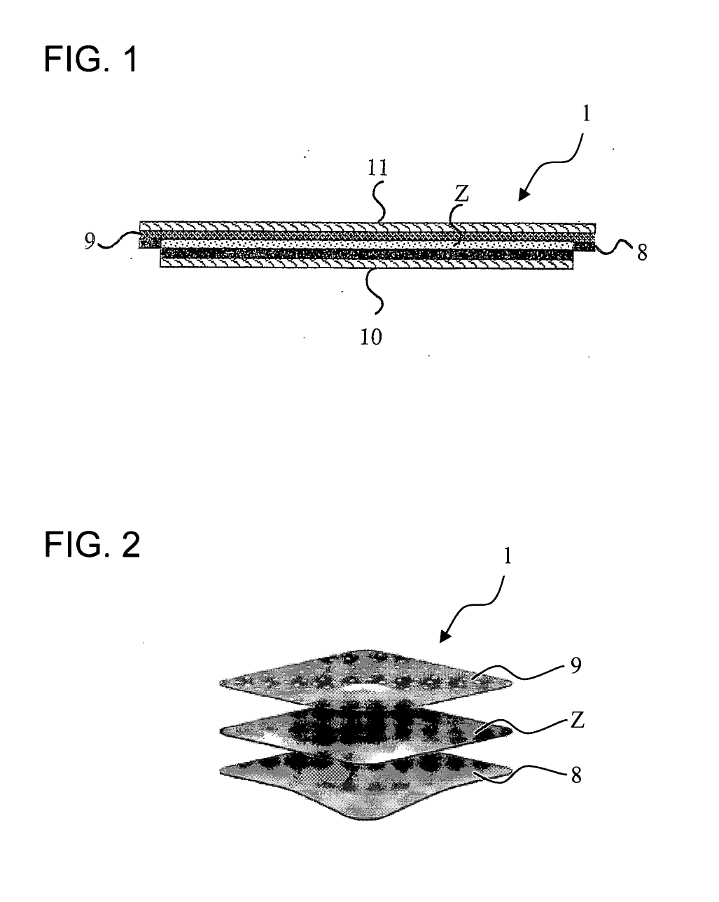

Referring now to the figures of the drawing in detail and first, particularly, to FIG. 1 thereof, there is shown a preferred embodiment of the method according to the invention. The patch (1) comprises:
a layer (8) impermeable to the at least one gaseous vanillin compound,
the overlying layer (9), permeable to the at least one gaseous vanillin compound, and the composition (Z) located between both layers,
layer (8) impermeable to the at least one gaseous vanillin compound being provided on its side facing away from the permeable layer (9) with an adhesive,
a first, protective liner layer (10), which attaches adhesively to the side of the impermeable layer (8) that has been provided with the adhesive, and which can be separated from this film by peeling by hand, preferably through the action of a force of less than 10 N, more preferably of less than 1 N, and with further preference of less 0.1 N, the adhesive remaining on the impermeable layer (8),
a second protective liner layer (11), made of a material which is impermeable to the at least one gaseous vanillin compound, which attaches adhesively to the permeable layer (9) on its side that is facing away from the impermeable layer (8), and can be separated by peeling by hand, preferably by the action of a force of less than 10 N, more preferably less than 1 N and with further preference less than 0.1 N, from the permeable layer (9).

FIG. 2 shows the patch (1) according to FIG. 1 without the covering protective layers (10) and (11) or further additional packagings.

When the patch (1) is brought into the vicinity of the body, by means for example of the adhering of the patch to the skin of the wearer, following the removal of the first protective layer (10), the impermeable layer (8) entering into contact with the skin, it permits the continuous evaporation of the composition and hence the uptake of the at least one gaseous vanillin compound, through the nose and/or the lungs of the wearer. This is made possible by virtue of the fact that, owing to the body temperature of the wearer, the evaporation of the composition in the patch and hence also the passage of the at least one gaseous vanillin compound, through the permeable layer (9), after the second protective liner layer (11) has been removed, is promoted.

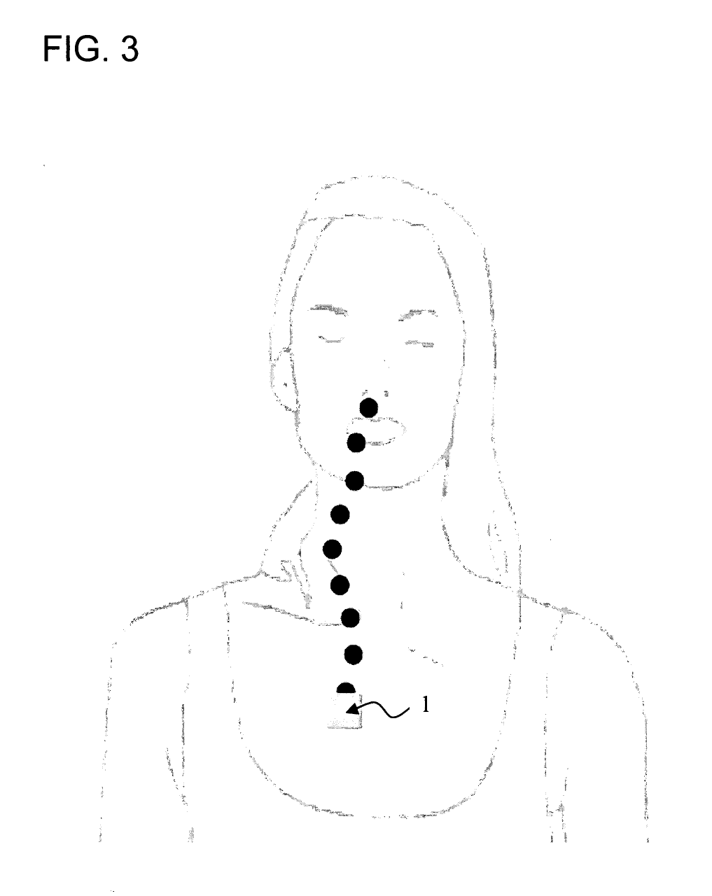

FIG. 3 finally shows the patch (1) according to FIG. 2, adhered to the skin in the area of the décolleté of a human wearer. In the preferred application, the patch is directly adhered to the skin of the person below the head, such as on the neck of the person or on the chest.

The patch (1) according to the invention renders possible a continuous absorption of vanillin, ethylvanillin or mixtures of those through the nasal or pulmonary way (indicated in FIG. 3 by dots).

It is preferred for this patch (1) not to comprise any lint or similar materials impregnated with the composition. In this context it is further preferred for the composition to have a dynamic viscosity in range from 10² to 10⁶, more preferably in a range from 10³ to 10⁵ mPa·s at 20° C. in accordance with DIN 53211.

The adhesive present in the device of the invention preferably comprises those adhesives known to the skilled worker that can be used to attach a patch to the skin of a wearer. Preferred adhesives are those which are offered by Dow Corning Corporation, USA, under the commercial designation BIO PSA, and also acrylate-based or silicone-based adhesives which are approved for medical use.

The protective liner layers are preferably films which comprise the materials listed in connection with the impermeable layer. The thickness of these protective layers as well corresponds preferably to that of the permeable or impermeable layer. Particularly preferred protective layers used are films comprising polyethylene or polypropylene.

The purpose of the protective layers attaching adhesively to the permeable layer is to prevent evaporation of the composition from the device before it is used for the controlled release of the at least one gaseous vanillin compound. These protective layers must, accordingly, be impermeable to the at least one gaseous vanillin compound. For this reason they preferably have a thickness such that not more than 1%, preferably not more than 0.1%, and more preferably not more than 0.01% by weight of the composition is able to evaporate via this protective layer within a time interval of 24 hours at room temperature under a pressure of 1 bar.

The composition comprised in the device of the invention is based preferably on:
(α1) the at least one gaseous vanillin compound, in an amount in a range from 0.1% to 100% by weight, preferably in a range from 1% to 50% by weight, and with further preference in a range from 5% to 20% by weight,
(α2) a vehicle in an amount in a range from 0% to 99% by weight, more preferably in a range from 20% to 70% by weight, and with further preference in an amount in a range from 40% to 60% by weight, and
(α3) excipients in an amount in a range from 0% to 50% by weight, more preferably in an amount in a range from 10% to 40% by weight, and with further preference in an amount in a range from 20% to 30% by weight,
   the sum of components (α1) to (α3) being 100% by weight.

The term “vanillin compound” is used to refer to the chemical substance vanillin, 3-methoxy-4-hydroxybenzaldehyde and its isomers and derivatives in which one or more hydrogen atoms are substituted by nitro groups or lower alkyl groups having one to three carbon atoms. Preferred vanillin compounds are vanillin, ethylvanillin, 3-ethoxy-4-methoxybenzaldehyde, and isovanillin, 4-methoxy-3-hydroxybenzaldehyde.

The vehicle preferably comprises monohydric alcohols, such as ethanol, polyhydric alcohols, such as glycerol, ethylene glycols or their ether derivatives, such as diethylene glycol, polyethylene glycol, diethylene glycol diethyl ether, polyethylene glycol dimethyl ether, essential oils, examples being cedar wood oil, cedar leaf oil, thuja oil, lavender oil, lavandin oil, lemon oil, Litsea cubeba oil (may chang oil) or verty oil, silicone oils, waxes, fats, such as olive oil, groundnut oil or coconut fat, or mixtures of at least two of the aforementioned vehicles. The vehicle can further comprise resinous and rubbery materials, such as methylcellulose, ethylcellulose, hydroxypropyl methylcellulose, natural rubber, butadiene-styrene copolymer rubber, and ethylene-propylene copolymer rubber.

Excipients present are preferably substances which promote the evaporation of the at least one gaseous vanillin compound. Preferred evaporation-promoting excipients are selected from the group consisting of hexyl acetate, isobutyl acetate, cyclohexyl acetate, prenyl acetate, isononyl acetate, isobornyl acetate, linalyl acetate, benzyl acetate, ethyl butyrate, camphor, ethyl caproate, methyl caproate, carenene, limonene, the terpene oil from oranges, allyl heptanoate, methyl hexenoate, ethyl isobutyrate, methyl pentyl ketone, myrcene, phellandrene, pinene, butyl valerate, terpinolene, liquid paraffins or mixtures of at least two of these compounds. Preferred excipients are, furthermore, dyes, preservatives, viscosity regulators, UV stabilizers, and further aromatic compounds, coumarin for example. Further-preferred excipients are fragrances other than the at least one gaseous vanillin compound, preferably selected from the group consisting of orange, neroli, jasmine, balm, sandalwood, bergamot, oak moss, eucalyptus, fennel, camomile, caraway, sage, rosemary, star anise, tea tree oil, incense, cinnamon bark, stone pine, almond oil, jojoba or mixtures of at least two thereof. If the composition is used in a device for trans-dermally administering the at least one gaseous vanillin compound, then the composition preferably also includes, as excipients, penetration promoters, examples being mixtures of 1 menthol and propylene glycol.

As the composition it is preferred to use a mixture of floral vanillin (ref. C6031/E Flora from A. Algto Ltd) as the at least one vanillin compound and ethanol. In order to increase the viscosity it is possible for this mixture further to comprise viscosity regulators, glycol for example.

In a particularly preferred embodiment, the composition suitable for the device of the invention shaped as a patch is formulated with 1-5% by weight of the vanillin compound, 10-30% by weight synthetic rubber Ecomelt T8 EX 24 (Source: Colano Co., Switzerland), 50-75% by weight synthetic rubber Durotak H 112 (Source: National Starch Co., USA), 1-5% by weight ethylcellulose N50 NF, 1-5% by weight glycerol, and 1-5% propylene glycol, provided that the total of al ingredients is 100% by weight.

The invention also relates to a process for producing the above-described device for controlledly releasing the at least one gaseous vanillin compound.

If the device is shaped in the form of a patch, then the device of the invention is produced preferably by introducing the composition between the permeable layer and the impermeable layer. This is preferably accomplished by first applying the composition to a defined area of the permeable or impermeable layer and placing the other layer over the composition, the permeable and impermeable layers being connected to one another at those points at which they directly contact one another. This joining takes place preferably by welding or adhesive bonding.

The invention further relates to the devices obtainable by processes described above.

The invention also relates to the use of the at least one gaseous vanillin compound, to regulate the craving to consume food.

The present invention also provides for the use of an above-described device for the nasal or pulmonary uptake of the at least one gaseous vanillin compound, to stimulate the release of serotonin in the central nervous system.

Finally the present invention also relates to the use of an above-described device in a dietary regime or in combination with foods selected for a particular diet. For example, the device of the invention is used together in a food-combining diet in combination with foods suitable for the diet and containing only protein or carbohydrates. In another diet, the device of the invention is used with liquid food such as fruit and vegetable juices.

The invention is now illustrated with reference to a non-limiting example.

### EXAMPLES

A method according to the invention in shape of a patch is designed in such a way that, after adhering the patch to the human skin, preferably in the area of the chest or décolleté, continuously and at constant rate at least during the normal wake period of a person, thus at least during 18 hours, preferably however during a whole day, thus day and night during 24 hours, the precious substances of odor and flavor implied in the interface layer Z are dispensed.

Very soon the wearer of the patch according to the invention reaches a maximization of the olfactory saturation, which after approximately 4-6 days passes into a cumulation of odors and flavors in the limbic system.

The permanent supply to the nose (nervus olfactorius) and to the limbic system effects a maximization of the olfactory saturation (supersaturation) and at the same time a supersaturation of the limbic system.

This leads to a reduction of ravenousness paired with the decrease of eating and munching attacks. Additionally a clear reduction of appetite for sweets like chocolate or “noshes” like chips etc. can be observed.

In the case of a group of 150 adult test persons having a body mass index of between 25 and 27, over a wearing period of 45 days per unit, a reduction in food consumption and eat-attacks was observed over a period of 31 days. The test persons lost on average about 0.4 kg per day.

I claim:

1. A method of assisting a person in need thereof to regulate the craving to consume food, which comprises:
providing a patch formed with a first layer substantially impermeable to a gaseous vanillin compound, a second layer permeable to the gaseous vanillin compound, and a composition comprising at least one vanillin compound disposed between the first and second layers;
the patch not comprising lint impregnated with the composition;
placing the patch on the person substantially below a head region with the first layer facing towards the person's body, and continuously releasing the gaseous vanillin compound through the second layer for nasal or pulmonary uptake by the person wearing the patch to reduce a craving to consume food.

2. The method according to claim 1, which comprises adhering the patch to the person's skin in a chest area.

3. The method according to claim 1, which comprises providing the patch with fully compliant first and second layers not including a resilient portion.

4. The method according to claim 1, wherein said at least one vanillin compound is selected from the group consisting of vanillin and ethylvanillin.

5. The method according to claim 1, further comprising a first, protective liner layer attached adhesively to the side of said impermeable layer, and a second protective liner layer, which is impermeable to the at least one gaseous vanillin compound, attached adhesively to the permeable layer on its side that is facing away from the impermeable layer.

6. The method according to claim 1, wherein the composition has a dynamic viscosity in a range from 10² to 10⁶ mPa·s at 20° C. in accordance with DIN 53211.

7. The method according to claim 6, wherein the composition has a dynamic viscosity in a range from 10³ to 10⁵ mPa·s at 20° C. in accordance with DIN 53211.

8. The method according to claim 1, wherein the composition is based on:
(α1) vanillin and/or ethyl vanillin in an amount in a range from 0.1% to 100% by weight;
(α2) a vehicle in an amount in a range from 0% to 99% by weight; and
(α3) excipients in an amount in a range from 0% to 50% by weight;
a sum of components (α1) to (α3) being 100% by weight.

9. The method according to claim 8, wherein said vehicle comprises synthetic rubber.

10. The method according to claim 1, which comprises adjusting a thickness of the first layer impermeable to the at least one gaseous vanillin compound such that within a time interval of 24 hours not more than 1% by weight of the composition can evaporate via this film at room temperature under a pressure of 1 bar.

11. The method according to claim 10, wherein said layer has a thickness in a range from 0.001 to 1 mm.

12. The method according to claim 1, which comprises adjusting the second layer permeable to the gaseous vanillin compound with a permeability to allow, within a time interval of 18 to 24 hours, at least 10% by weight of the at least one vanillin compound present in the composition to evaporate at room temperature under a pressure of 1 bar.

13. The method according to claim 12, wherein the permeable layer comprises a film comprising polyethylene, polyamide, ethylene-vinyl acetate copolymer or mixtures thereof.

14. The method according to claim 12, wherein said permeable layer has a thickness in a range from 0.001 to 1 mm.

15. The method according to claim 1, wherein the permeable layer is a film formed with holes having a diameter in a range between 1 and 100 Å.

16. The method according to claim 1, which comprises forming the second layer as a film with holes and a given density, such that within a time interval of 24 hours at room temperature under a pressure of 1 bar at least 10% by weight of the vanillin and/or ethyl vanillin present in the composition is able to evaporate.

17. The method according to claim 16, wherein the film is adjusted such that within a time interval of 24 hours at room temperature under a pressure of 1 bar at least 25% by weight of the vanillin and/or ethyl vanillin present in the composition is able to evaporate.

18. The method according to claim 16, wherein the film is adjusted such that within a time interval of 24 hours at room temperature under a pressure of 1 bar at least 50% by weight of the vanillin and/or ethyl vanillin present in the composition is able to evaporate.

19. The method according to claim 1, wherein said impermeable layer comprises aluminum foil.

20. A method of stimulating a release of serotonin in the central nervous system of a person, the method which comprises:
providing a patch formed with a first layer substantially impermeable to a gaseous vanillin compound, a second layer permeable to the gaseous vanillin compound, and a composition comprising at least one vanillin compound disposed between the first and second layers, the patch not comprising lint impregnated with the composition;
placing the patch on the person substantially below a head region with the first layer facing towards the person's body, and continuously releasing the gaseous vanillin compound through the second layer for nasal or pulmonary uptake by the person wearing the patch to influence a serotonin exchange in the central nervous system of the person.
